(19) United States
(12) Patent Application Publication
(10) Pub. No.: US 2026/0365597 A1
(43) Pub. Date: Nov. 19, 2026

(54) PRODUCTION OF AN OLEFIN BY DEHYDROGENATION OF A PARAFFIN

(71) Applicant: LINDE GMBH, Pullach (DE)

(72) Inventors: Rory LEWIS, Pullach (DE); Markus BERG, Pullach (DE)

(21) Appl. No.: 19/133,494

(22) PCT Filed: Oct. 31, 2023
(86) PCT No.: PCT/EP2023/025454

§ 371 Date: May 28, 2025

(30) Foreign Application Priority Data
Nov. 29, 2022 (EP) 22020582.7

(51) Int. Cl. C07C 5/333; B01J 19/24; C07C 7/00; C07C 7/04; C07C 7/09; F25J 3/06

(52) U.S. Cl. CPC C07C 5/333; B01J 19/24; C07C 7/005; C07C 7/04; C07C 7/09; F25J 3/06; B01J 2219/00103

(57) ABSTRACT

A process for producing an olefin using a catalytic dehydrogenation, having a mode of production operation which comprises subjecting a feed mixture containing a paraffin of equal chain length to the olefin to catalytic dehydrogenation to obtain a product mixture containing an unconverted portion of the paraffin, the olefin, and higher-boiling components than the olefin, subjecting the product mixture or a portion thereof to a condensate separation to obtain one or more condensates containing at least a portion of the paraffin, of the olefin and of the higher-boiling components than the olefin, subjecting the one or more condensates or a portion thereof to a flash evaporation to leave a residual liquid containing at least a portion of the paraffin and of the olefin from the one or more condensates, subjecting the residual liquid or a portion thereof to a heat transfer with heating.

The invention relates to a process and a plant for producing an olefin by dehydrogenating a paraffin.

### BACKGROUND

Processes for the dehydrogenation of paraffins, in particular propane to propylene, are known and described, for example, in the article “Propene” in Ullmann's Encyclopedia of Industrial Chemistry, online edition, Jun. 15, 2000, DOI 10.1002/14356007.a22_211, Section 4.3, “Propane Dehydrogenation”.

For example, reactors that have combustion chambers that are heated by burners and through which a plurality of reaction tubes are passed are used to dehydrogenate propane to propylene. The reaction tubes are externally heated by the burners. The reaction tubes contain a catalyst on a suitable carrier. Suitable catalysts are described, for example, in Bölt, H., “Dehydrierung von Propan/Butan” [“Dehydrogenation of Propane/Butane”], Linde Reports of Science and Technology 66/1991. The propane to be dehydrogenated or a propane-rich stream is passed through the reaction tubes together with steam, so-called process steam. This is typically preheated beforehand using waste heat. A gas mixture taken from the reactor or corresponding reaction tubes, hereinafter also referred to as a product mixture, is fed to product processing.

The dehydrogenation of propane to propylene is highly selective when choosing suitable catalysts and reaction conditions and can be over 90%. However, hydrogen that is split off during dehydrogenation and small amounts of by-products in the form of methane and other hydrocarbons are always produced. Furthermore, in a corresponding product mixture, this is always larger amounts of propane that have not been converted into propylene and process steam.

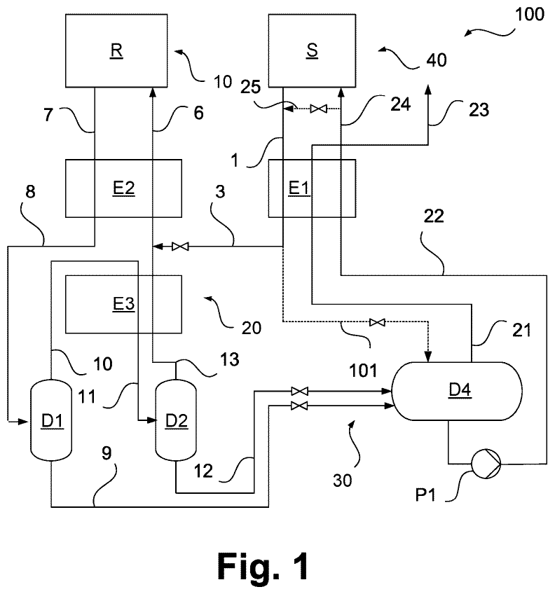

Examples of corresponding product processing are illustrated in FIGS. 1 and 2 in EP 3 379 187 A1. For further details, reference is made in particular to paragraphs [0025] to [0030] therein. In a corresponding arrangement, a low-temperature separation unit referred to in EP 3 379 187 A1 as a cryogenic treatment device is used.

A corresponding low-temperature separation unit together with its various apparatuses is typically at least partially arranged in a so-called cold box. The term “cold box” is usually understood to mean a thermally insulating enclosure in which processing apparatuses operated at low, in particular cryogenic, temperatures are installed. In a cold box, a plurality of apparatuses, i.e., for example, a plurality of columns, separators and heat exchangers, can be fastened together with the associated piping to a supporting steel frame which is externally covered with sheet metal plates. The interior of the enclosure formed in this way is filled with insulating material, such as perlite, in order to prevent heat input from the environment.

The object of the present invention is, among other things, to provide solutions that allow a corresponding low-temperature separation unit or cold box to be put into operation as quickly and smoothly as possible without exceeding the permissible temperature gradients in terms of time and location. In particular, warm-up scenarios and (possibly due to extreme climatic conditions) starting up in winter should be taken into account.

### DISCLOSURE OF THE INVENTION

Against this background, the present invention proposes a process and a plant for the production of olefins by dehydrogenating a paraffin having the features of the independent claims. Each of the embodiments are the subject matter of the dependent claims and of the description below.

The present invention in particular allows for a more gentle start-up of a corresponding plant for the production of olefins with product processing. In the process, in particular before starting the actual production process, the heat exchanger that is used for heating a hydrocarbon fraction that contains unconverted paraffin and the formed olefin can advantageously be cooled. In addition to this independent temperature reduction in preparation of the production process, a further aspect and advantage of the present invention consists in setting or maintaining an acceptable temperature gradient in said heat exchanger by setting a “start-up propane circuit”, as explained in more detail below as part of a two-stage process or model.

The specific advantages of the present invention are revealed in particular in connection with the dehydrogenation of propane to propylene and will be described below mainly with reference thereto. In principle, however, it can also be used in connection with other dehydrogenation processes, for example to dehydrogenate butane.

Take, for example, FIG. 1 of EP 3 379 187 A1 (the following reference signs initially refer to this figure) in which a flash-evaporation of condensates from the vessels D1 and D2 into a flash vessel D4 is performed.

In a production mode, the paraffin fed to the reaction unit R in the form of the material stream 6 can be taken from a paraffin separation process as a material stream 1 to which process a material stream 22 or 24 from the vessel D4 containing the paraffin and the corresponding olefin is, in turn, fed, which stream is taken from the vessel D4 by means of the pump P. However, this is only possible if corresponding condensates are present in the vessels D1 and D2, i.e. the product mixture 7 has been formed over a sufficiently long period of time. If this is the case, a corresponding paraffin cycle is created.

As long as there are still no condensates in the vessels D1 and D2, the corresponding feed also cannot be supplied to corresponding paraffin separation. Therefore, the paraffin must first be supplied from another source. In this case, however, the heat exchanger E1 has not yet cooled down. However, this is irrelevant for loading the reaction unit R.

As condensation begins, condensate first collects in the vessels D1 and D2, but warm paraffin must continue to be supplied until the paraffin separation produces sufficient paraffin. This results in considerable thermal stresses in the heat exchanger E1. Once sufficient fill levels have been reached in the vessels D1 and D2 and the expansion vessel D4, the aforementioned circuit can be started via the pump P1. This means that cold liquid from the pump P1 strikes the still-warm exchanger E1 and also causes high thermal stresses.

This is where the invention comes in. It allows the temperature of the heat exchanger E1 to be gradually controlled before the beginning of the production process and subsequently allows for adjustment or maintenance of an acceptable temperature gradient in said heat exchanger E1. The present invention is not limited by the above reference to EP 3 379 187 A1.

Against this background, a process for producing an olefin using catalytic dehydrogenation is proposed. In this case, a production operating mode is performed which involves subjecting a feed mixture that contains a paraffin having the same chain length as the olefin to catalytic dehydrogenation to obtain a product mixture which contains an unconverted part of the paraffin, the olefin, and higher boiling components than the olefin.

The product mixture or a portion thereof is subjected to condensate separation to obtain one or more condensates which contain(s) at least a portion of the paraffin, the olefin and higher boiling components than the olefin. The one or more condensates or a portion thereof is subjected to flash evaporation, leaving a residual liquid which contains at least a portion of the paraffin and the olefin from the one or more condensates. The residual liquid or a portion thereof is subjected to heat transfer while heating and then subjected to paraffin separation to obtain a paraffin fraction and an olefin fraction. The paraffin fraction or a portion thereof is subjected to heat transfer while cooling and then subjected to catalytic dehydrogenation once again.

A special operating mode is proposed which can be carried out in particular after (re-)starting a corresponding plant or before the production operating mode, and which involves expanding a cryogenic liquid containing the paraffin into an evaporation vessel which is used for flash evaporation, and passing flash gas formed thereby through a heat exchanger used for heat transfer. In this way, pre-cooling the heat exchanger used for heat transfer (which may correspond to the heat exchanger E1 according to EP 3 379 187 A1) is possible. By feeding liquid paraffin and expanding it in the evaporation vessel (the temperature can be adjusted by means of the selected pressure), the formed flash gas can set the required temperature gradient by means of the heat exchanger. The aforementioned evaporation vessel is used as a flash or outgassing vessel in the production operating mode.

By using the present invention, the above-mentioned object, which consists in particular in putting the cold parts of a corresponding plant into operation as quickly and smoothly as possible without exceeding the temperature gradients with respect to time and location, can be achieved. In addition, the aforementioned warm-up scenarios (possibly due to extreme climatic conditions) and starting up in winter can be taken into account.

Without the proposed measures, the temperature gradients required during start-up/shutdown are much more difficult to achieve, and the cooling rate is considerably slower. The invention thus also allows for startup under extreme winter conditions and the operation of a corresponding plant during very hot periods. In such cases, it may be common for the design temperature of the inlet to be exceeded, which may lead to a shutdown.

Overall, a corresponding plant can be started up more quickly and simply by using the proposed measures according to the invention, which can be implemented in particular by using an additional control valve for expansion into the expansion vessel. The cold box can be cooled in such a way that flash vessels and liquid product pumps can be cooled separately from other components. This results in a gentler and more controlled cooling, Given a possible control of pressure in the flash vessel, the start of paraffin injection into the combined feed can be at a lower pressure.

Within the scope of the present invention, the temperature profile in the aforementioned heat exchanger can be adjusted so that unnecessary thermal loads are avoided. This may also allow the liquid product pump to be operated in bypass mode (using the power input as an evaporator) to supplement the gaseous propane supply required to adjust the composition of the feed discharge.

The liquid injection proposed in embodiments of the invention, i.e. the expansion of the liquid paraffin stream into the expansion vessel, enables early liquid accumulation in the upstream vessels and simplifies and accelerates cooling of the cold box. The liquid injection allows the supply of warm feed even when the cold box is under extreme winter conditions. In this way, the cold box can be pressurized in winter so that warm feed gas can be introduced later on.

Through optimal liquid injection, the feed temperature into the cold box can be reduced by about 2 to 5 °C. This may be necessary in hot summer conditions in warm climates (risk of exceeding the design temperature of the cold box).

Once again, in summary a smoother and more even start-up can minimize thermal loading during start-up and normal operation, which leads to a longer service life of the involved (plate) heat exchangers. Start-up and normal operation can take place under extreme climatic conditions (summer and winter), which leads to lower thermal loading during start-up and normal operation and thus to a longer service life of the heat exchangers.

Embodiments of the present invention may include forming the paraffin-containing cryogenic liquid using the paraffin fraction or a portion thereof and/or using externally supplied paraffin. In particular, this can be “make-up” paraffin which is also used in the production mode to compensate for the proportion of paraffin converted during catalytic dehydrogenation each time.

In one embodiment of the invention, after heating, an adjustable proportion of the residual liquid is fed to the paraffin fraction before being cooled, bypassing paraffin separation, and is subjected to cooling together therewith and then subjected to catalytic dehydrogenation once again. In this way, a corresponding “start-up propane circuit” can be created which allows an adjustment or maintenance of the temperature profile in the heat exchanger used for heating or cooling.

Within the scope of the present invention, condensate separation may comprise a first cooling step from a first temperature level to a second temperature level below the first temperature level and the subsequent separation of a first condensate, as well as a second cooling step from the second temperature level to a third temperature level below the second temperature level and the subsequent separation of a second condensate. In this context, the present invention can be used in particular with tried-and-tested product processing technologies, as already explained with reference to the prior art.

More specifically, a gas fraction or a portion thereof remaining when the first condensate is separated may be subjected to the separation of the second condensate, and a gas fraction or a portion thereof remaining when separating the second condensate may be used as a refrigerant for the first cooling step and/or the second cooling step.

In particular, flash evaporation may be carried out at the second temperature level, the residual liquid or the portion thereof that is subjected to paraffin separation may be heated from the second temperature level to the first temperature level using heat transfer, and the paraffin fraction or the portion thereof that is again subjected to catalytic dehydrogenation may be cooled from the first temperature level to the second temperature level using heat transfer.

Embodiments of the present invention include, in particular, the paraffin fraction or the portion thereof that is returned to dehydrogenation being used as a refrigerant for the first cooling step.

In embodiments of the present invention, the first temperature level can be 0 to 50 °C, in particular 25 to 35 °C, the second temperature level can be −50 to 0 °C, in particular −40 to −45 °C, for example approximately 42 °C, and the third temperature level can be −150 to −100 °C, in particular −110 to 130 °C, for example 120 °C.

As mentioned, the paraffin may be propane and the olefin may be propylene, and the higher boiling components than the olefin may include, in particular, hydrogen and methane.

At the early stage of plant operation in which the present invention is used according to various embodiments, there are typically no higher-boiling components in the plant except for nitrogen left over from inerting. However, this nitrogen is immediately reduced to an insignificant residual concentration by dilution. Typically, so-called raw propane is therefore present which, for example, has a propane content of more than 95 or 99 mole percent. The expansion, which defines the temperature of the flash gas, is accordingly adjusted depending on the pressure, as already mentioned above. The cooling of this region is completed with the formation of a condensate phase (two-phase equilibrium). Especially with propane, the required temperature can be easily adjusted.

In embodiments of the present invention, the special operating mode can be performed on the basis of a temperature measurement on or in at least one apparatus used in the process, wherein the special operating mode is terminated when a temperature value determined when measuring the temperature falls below a predetermined threshold value, or can be terminated when the production operating mode provides a sufficient amount of condensates from the utilized separators.

A plant for producing an olefin that comprises a reactor unit, a condensate separation unit, a flash evaporation unit and a paraffin separation unit is also proposed. The plant is designed to perform a production operating mode in which the reactor unit is used to subject a feed mixture that contains a paraffin with the same chain length as the olefin to catalytic dehydrogenation to obtain a product mixture that contains an unconverted portion of the paraffin, the olefin, and higher boiling components than the olefin, the product mixture or a portion thereof is subjected to condensate separation using the condensate separation unit to obtain one or more condensates that contain at least a portion of the paraffin, the olefin, and the higher boiling components than the olefin, the flash evaporation unit is used to subject the one or more condensates or a portion thereof to flash evaporation, leaving a residual liquid that contains at least a portion of the paraffin and the olefin from the one or more condensates, the residual liquid or a portion thereof is heated and subjected to heat transfer and then subjected to paraffin separation using the paraffin separation unit to obtain a paraffin fraction and an olefin fraction, and the paraffin fraction or a portion thereof is cooled and subjected to heat transfer and then to catalytic dehydrogenation once again.

A plant according to the invention is designed to perform a special operating mode and, in this mode, to expand a paraffin-containing cryogenic liquid into an evaporation vessel in the flash evaporation unit and to pass a flash gas formed during this expansion process through a heat exchanger used for the aforementioned transfer of heat.

For further features and advantages of a corresponding plant and embodiments thereof, reference is expressly made to the above explanations relating to the process proposed according to the invention and its embodiments, since they apply in the same way here.

The same also applies to a plant which, according to one embodiment of the invention, is configured to carry out a process according to any embodiment of the present invention.

### BRIEF DESCRIPTION OF THE DRAWING

Embodiments of the invention will be described below purely by way of example and with reference to the accompanying drawings, in which FIG. 1 illustrates a plant according to one embodiment of the invention.

### EMBODIMENTS OF THE INVENTION

The embodiments described below are described solely for the purpose of assisting the reader in understanding the features claimed and previously discussed. They are merely representative examples and are not intended to be considered to be exhaustive and/or limiting with respect to the features of the invention. It goes without saying that the advantages, embodiments, examples, functions, features, structures and/or other aspects described above and below are not to be considered to limit the scope of the invention as defined in the claims, or to limit equivalents to the claims, and that other embodiments may be used and changes made without departing from the scope of the claimed invention.

Different embodiments of the invention may comprise, have, consist of, or substantially consist of further expedient combinations of the described elements, components, features, parts, steps, means, etc., even if such combinations are not specifically described herein. Furthermore, the disclosure may comprise other inventions that are presently not claimed, but which may be claimed in the future, in particular when included in the scope of the independent claims.

Explanations relating to devices, apparatuses, arrangements, systems, etc. according to embodiments of the present invention may also apply to procedures, processes, methods, etc. according to the embodiments of the present invention, and vice versa. Elements, process steps, etc. that are identical, have the same effect, correspond in terms of their function, are structurally identical or have a similar structure can be indicated by identical reference signs.

The present invention and embodiments thereof are explained below with reference to electrolysis with a proton exchange membrane. As mentioned several times, however, the invention is not limited to this.

FIG. 1 illustrates a plant according to one embodiment of the present Invention and is designated as a whole by reference sign 100. The reference signs from EP 3 379 187 A1 are used below without the intent of a corresponding restriction.

In the plant 100 process, a feed mixture 6, which contains propane, for example, is subjected to catalytic dehydrogenation in a reaction unit 10 having a reactor R to obtain a product mixture 7 which contains an unconverted portion of the propane, propylene, and higher boiling components than the propylene, in particular hydrogen and methane.

The product mixture 7 is passed through a heat exchanger E2 in a condensation unit designated as a whole by 20 and then fed into a vessel D1 in the form of a material stream 8. A first condensate is separated in the vessel D1 and fed in the form of a material stream 9 for flash evaporation in a flash vessel D4. The flash evaporation is indicated as a whole by reference sign 30.

A portion remaining in gaseous form in the vessel D1 is further cooled in a heat exchanger E3 in the form of a material stream 10 and then fed into a vessel D2 in the form of a material stream 11. A second condensate is separated in the vessel D2 and is also fed in the form of a material stream 12 for flash evaporation 30 in the flash vessel D4.

A portion remaining in gaseous form in the vessel D2 is heated in the heat exchangers E3 and E2 in the form of a material stream 13, combined with a propane stream 3 therebetween, and returned to the reaction unit 10.

During flash evaporation, a residual liquid remains in the flash vessel D4 which contains at least a portion of the paraffin and the olefin from the one or more condensates. This is passed through a heat exchanger E1 by means of a pump P1 in the form of a material flow 22 where it is subjected to heat transfer while being heated. Subsequently, a feed in the form of a material stream 24 takes place a paraffin separation, designated by 40 on the whole, with a separation unit S.

Flash gas from the expansion vessel D4 can also be passed through the heat exchanger E1 in the form of a material stream 21 and discharged from the process or plant 100 in the form of a material stream 23.

During paraffin separation 40, a paraffin fraction, here a propane fraction, and an olefin fraction, here a propylene fraction, are formed, wherein the paraffin fraction in the form of a material stream 1 is cooled in the heat exchanger E1 and then used to form the propane stream 1, le. is again subjected to catalytic dehydrogenation 10 in the reaction unit R.

As illustrated by a dashed flow arrow, in the embodiment of the invention illustrated here, a special operating mode is provided which involves a cryogenic liquid containing the paraffin, in this case propane, as illustrated by a material stream 101, being expanded into the evaporation vessel D4 that is used during flash evaporation 30, wherein the flash gas formed during this expansion as well as the “regular” flash gas in the form of the material stream 21 is passed through a heat exchanger E1 used for the aforementioned transfer of heat.

At any time, i.e. including during regular operation, in the process illustrated here or a corresponding plant 100, an adjustable proportion of the residual liquid, i.e. of the material stream 22 after being heated in the heat exchanger E1, can be fed to the paraffin fraction or the material stream 1 before it is cooled in the heat exchanger E1, while bypassing paraffin separation S, and subjected to cooling together with said fraction or material stream, and then again subjected to catalytic dehydrogenation. As a result, the frequently mentioned “start-up propane circuit” may be realized with corresponding advantages, and the temperature of the heat exchanger E1 can thus be controlled.

1. A process for producing an olefin using catalytic dehydrogenation, comprising a production operating mode that involves:
a feed mixture that contains a paraffin having the same chain length as the olefin being subjected to catalytic dehydrogenation to obtain a product mixture, which contains an unconverted portion of the paraffin, the olefin, and higher boiling components than the olefin,
subjecting the product mixture or a portion thereof to condensate separation to obtain one or more condensates which contain(s) at least a portion of the paraffin, the olefin and higher boiling components than the olefin,
subjecting the one or more condensates or a portion thereof to flash evaporation to leave a residual liquid which contains at least a portion of the paraffin and the olefin from the one or more condensates,
subjecting the residual liquid or a portion thereof to a heat transfer with heating and then to paraffin separation to obtain a paraffin fraction and an olefin fraction, and
subjecting the paraffin fraction or a portion thereof to a heat transfer with cooling and then again to catalytic dehydrogenation,
wherein a special operating mode involves
expanding a cryogenic liquid containing the paraffin into an evaporation vessel (D4) which is used in the flash evaporation, and guiding a flash gas formed during this expansion through a heat exchanger (E1) used for the aforementioned heat transfer.

2. The process according to claim 1, wherein the cryogenic liquid containing the paraffin is formed using the paraffin fraction or a portion thereof and/or using externally supplied paraffin.

3. The process according to claim 1, wherein, after heating, an adjustable proportion of the residual liquid is fed to the paraffin fraction before being cooled, bypassing the paraffin separation, and is subjected to cooling together therewith and then again to catalytic dehydrogenation.

4. The process according to claim 1, wherein condensate separation involves a first cooling step from a first temperature level to a second temperature level below the first temperature level and the subsequent separation of a first condensate, as well as a second cooling step from the second temperature level to a third temperature level below the second temperature level and the subsequent separation of a second condensate.

5. The process according to claim 4, wherein a gas fraction, or a portion thereof remaining during the separation of the first condensate, is subjected to the separation of the second condensate, and a gas fraction, or a portion thereof, remaining when the second condensate is separated is used as a refrigerant for the first cooling step and/or the second cooling step.

6. The process according to either claim 4, wherein the flash evaporation process is carried out at the second temperature level, wherein the residual liquid or the portion thereof that is subjected to paraffin separation is heated from the second temperature level to the first temperature level using heat transfer, and wherein the paraffin fraction or the portion thereof that is again subjected to catalytic dehydrogenation is cooled from the first temperature level to the second temperature level using heat transfer.

7. The process according to claim 4, wherein the paraffin fraction or the portion thereof that is returned to the dehydrogenation process is used as a refrigerant for the first cooling step.

8. The process according to claim 4, wherein the first temperature level is 0 to 50 °C, the second temperature level is −50 to 0 °C and the third temperature level is −150 to −100 °C.

9. The process according to claim 4, wherein the paraffin is propane and the olefin is propylene, and wherein the higher boiling components than the olefin include hydrogen and methane.

10. The process according to claim 1, wherein the special operating mode is performed on the basis of a temperature measurement on or in at least one apparatus used during the process, wherein the special operating mode is terminated when a temperature value determined when measuring the temperature falls below a predetermined threshold value.

11. A plant for producing an olefin, that has a reactor unit, a condensate separation unit, a flash evaporation unit and a paraffin separation unit, the plant being designed to perform a production operating mode in which
the reactor unit is used to subject a feed mixture that contains a paraffin with the same chain length as the olefin to catalytic dehydrogenation to obtain a product mixture that contains an unconverted portion of the paraffin, the olefin, and higher boiling components than the olefin,
the condensate separation unit is used to subject the product mixture or a portion thereof to condensate separation to obtain one or more condensates which contain at least a portion of the paraffin, the olefin, and the higher boiling components than the olefin,
the flash evaporation unit is used to subject the one or more condensates or a portion thereof to flash evaporation, leaving a residual liquid which contains at least a portion of the paraffin and the olefin from the one or more condensates,
the residual liquid or a portion thereof is heated and subjected to heat transfer and then subjected to paraffin separation using the paraffin separation unit and obtaining a paraffin fraction and an olefin fraction, and
the paraffin fraction or a portion thereof is cooled and subjected to heat transfer and then subjected to catalytic dehydrogenation once again,
wherein the plant is designed to perform a special operating mode in which
a cryogenic liquid containing the paraffin is expanded into an evaporation vessel (D4) in the flash evaporation unit, and a flash gas formed during this expansion is passed through a heat exchanger (E1) used for the aforementioned transfer of heat.

12. The plant according to claim 11, which is configured to carry out a process according to
a feed mixture that contains a paraffin having the same chain length as the olefin being subjected to catalytic dehydrogenation to obtain a product mixture, which contains an unconverted portion of the paraffin, the olefin, and higher boiling components than the olefin,
subjecting the product mixture or a portion thereof to condensate separation to obtain one or more condensates which contain(s) at least a portion of the paraffin, the olefin and higher boiling components than the olefin,
subjecting the one or more condensates or a portion thereof to flash evaporation to leave a residual liquid which contains at least a portion of the paraffin and the olefin from the one or more condensates,
subjecting the residual liquid or a portion thereof to a heat transfer with heating and then to paraffin separation to obtain a paraffin fraction and an olefin fraction, and
subjecting the paraffin fraction or a portion thereof to a heat transfer with cooling and then again to catalytic dehydrogenation,
wherein a special operating mode involves
expanding a cryogenic liquid containing the paraffin into an evaporation vessel (D4) which is used in the flash evaporation, and guiding a flash gas formed during this expansion through a heat exchanger (E1) used for the aforementioned heat transfer.
